(19) United States
(12) Patent Application Publication
(10) Pub. No.: US 2023/0420091 A1
(43) Pub. Date: Dec. 28, 2023

(54) INTERACTIVE ELECTRONIC HEALTH RECORD

(71) Applicant: Healthcare Integrated Technologies Inc., Sherwood, OR (US)

(72) Inventors: Stephen Jay Datena, Portland, OR (US); Jurgen Klaus Vollrath, Sherwood, OR (US)

(21) Appl. No.: 18/326,944

(22) Filed: May 31, 2023

Related U.S. Application Data
(60) Provisional application No. 63/473,737, filed on Jun. 22, 2022.

(51) Int. Cl. G16H 10/60; G16H 50/20; G06F 3/04817; G06F 3/0486

(52) U.S. Cl. CPC G16H 10/60; G16H 50/20; G06F 3/04817; G06F 3/0486

(57) ABSTRACT

An interactive EHR user interface that represents information graphically by depicting parts or systems of a body using images, and allowing persons to interact with the depictions by associating animations or icons with the depictions, and avoiding rejection of reimbursement requests by building billing codes into an AI system that defines the workflow during a patient encounter.

### FIELD OF THE INVENTION

The invention relates to Electronic Health Records.

### BACKGROUND OF THE INVENTION

A variety of electronic health records (EHRs) have been created, ostensibly to simplify record keeping for physicians and to provide for greater compliance and transparency of reimbursable procedures that are performed.

However, these EHR systems tend to simply be electronic versions of traditional paper records, involving pages of text defining the procedures, diagnoses, follow-ups, and orders associated with a patient encounter.

Furthermore, they are extremely time consuming, both in their every-day use and in the time spent by physicians in becoming familiar with the user interface and the features. Additional time is also spent by support staff in identifying reimbursement codes to apply, and payments are often denied for lack of documentary support.

### SUMMARY OF THE INVENTION

The present invention defines an interactive EHR interface. According to the invention, there is provided an EHR user interface (UI) that is intuitive to physicians by including depictions of at least one of: parts of the body, and systems of the body, and further including means for graphically representing information associated with said depictions of the parts or systems. For ease of reference the present description will describe the use with respect to a human body, and the claims will also refer to a human body. However, this is done to make the description and claims more intuitive and easier to understand since most applications will involve human applications. Nevertheless, the present invention is not so limited and could also be used for animals in a veterinarian environment. Thus, the term “human” is used herein for convenience and is intended to cover animals as well.

The depictions of the parts or systems may include depictions of one or more of a human torso, a depiction of a human skeleton, a depiction of a human head, depiction of a human nervous system, a depiction of a human digestive system, and a depiction of a human circulatory system. One or more of the depictions of the parts and systems may include visually defined regions that a physician can interact with. The interactions may take the form of auditory data captured using a digital medical device or sensor, e.g., a digital stethoscope or other device, and visually depicting the results associated with each region.

The graphical representation of information that can be associated with said depictions of the parts or systems may include animations that can be associated with specific regions on the depictions of the parts or systems.

The process for graphically interacting with the depictions in order to associate the graphic information with said depictions of the parts or systems may include selecting an animation (also referred to herein as an icon) from a library of animation and associating it with a specific location or region on or near the depiction of the body part or system. The animations may be presented as a global library or in the form of separate libraries for each graphic depiction of a part or system.

The associating of the animation with the location or region may include using active controls to identify regions and corresponding animations, or by dragging and dropping the animation on the desired region. The associating may also be performed automatically by monitoring the interactions between a physician and a patient during an encounter. The monitoring may include capturing one or more of the video data and auditory data by means of one or more cameras and microphones to identify body regions or locations identified or interacted with by the patient or physician.

Another aspect of the invention includes defining the workflow for the physician by using an artificial intelligence (AI) diagnostics engine. This may include using machine learning in analytics based on CPT, ICD-9 and 10 codes and combining with third party symptomatic-diagnostic information, e.g., peer reviewed medical literature. Thus, the creating and training of predictive models is based inter alia on medical billing codes and may be further supplemented using data from physician-patient encounters. The identified possible diagnoses can be used to refine the work flow, and inherently create the supporting documentation for the billing code to avoid rejection of reimbursement submissions.

Risk factors identified by the AI diagnostics engine may be highlighted on the graphic depictions. All risk factors for a patient may also be represented graphically for quick review of the patient prior to an encounter, e.g., by generating postage stamp images of one or more depictions that identify a risk factor. These postage stamp images may be presented to a physician when a patient record is opened, each image being an active, clickable icon that opens a full image with highlighting of the risk factor, e.g., in a different color.

By using an interactive graphics interface, the patients can also interact with depictions to support any verbal concerns they may have prior to an encounter.

### BRIEF DESCRIPTION OF THE DRAWINGS

FIG. 1 one embodiment of a user interface page on an EHR portal of the invention,

FIG. 2 shows another embodiment of a user interface page on an EHR portal of the invention,

FIG. 3 shows yet another embodiment of a user interface page on an EHR portal of the invention, and

FIG. 4 shows yet another embodiment of a user interface page on an EHR portal of the invention.

### DETAILED DESCRIPTION OF THE INVENTION

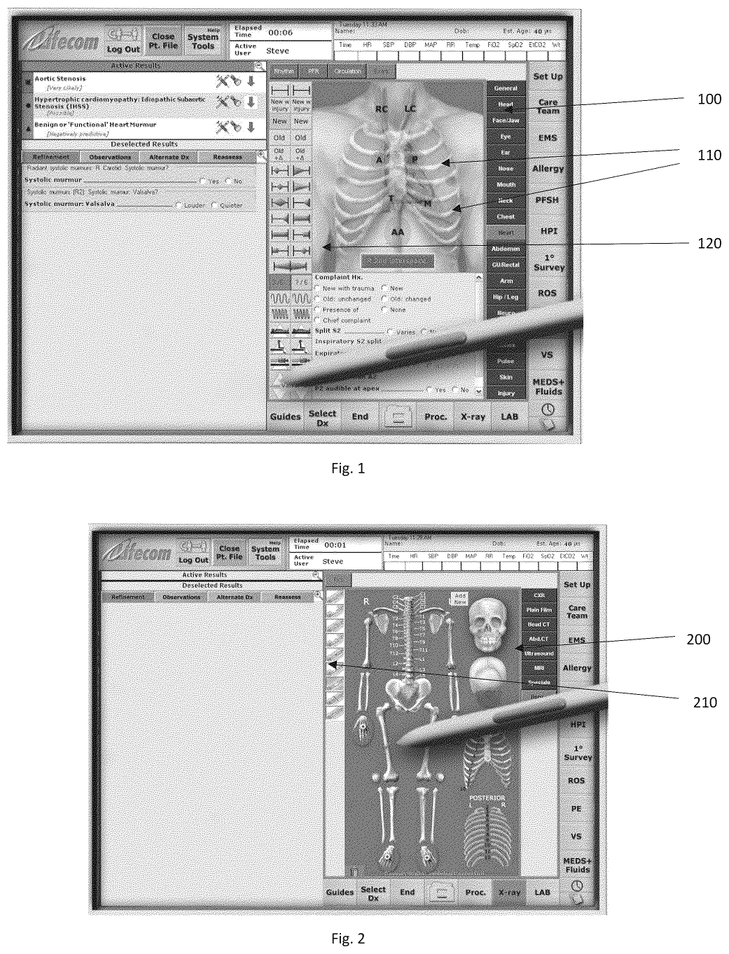

FIG. 1 shows one embodiment of a page of a user interface (UI) for an EHR system of the invention. The UI page of FIG. 1 shows a representation of a human torso 100 that includes multiple pre-defined regions 110 that a physician should analyze (e.g., by listening to the region with a stethoscope). By visually defining the regions, the UI inherently defines a workflow to ensure that critical regions are not inadvertently missed by the physician.

In this embodiment a library of animations 120 (also referred to herein as icons) are presented in a side-bar. By selecting a region being analyzed (e.g., by clicking on the region with the use of a mouse or touching the region on a touch-sensitive screen) and then selecting an animation 120 corresponding to the type of sound (e.g., early crescendo/decrescendo), the auscultation data associated with that region can be visually shown in relation to the torso depiction of the UI.

The library of animations that graphically define different auscultations with crescendo/decrescendo, thus allows the physician to graphically associate the type of stethoscope sound identified for each body region by relating an animation with each said region. In the above embodiment this is done by first clicking on the selected region on the torso and then clicking on the animation to be associated with the region (also referred to herein as using active controls).

In another embodiment, this association of animations with specific body regions may be performed using a touch-screen with drag-and-drop functionality, whereby animations can be dragged from a side bar to the associated region on the torso representation.

In yet another embodiment, the association of animations with body regions can be automated using a camera or microphone or both a camera and microphone that are time synched to a digital stethoscope to correlate the information and auto-associate each body region with the corresponding animation. The details for correlating image data captured by a camera or oral data captured by a microphone, with a body region for purposes of mapping data to a particular location on a user interface, is discussed in commonly owned U.S. utility application Ser. No. 17/499,412 entitled Electronic Health Record System and Method, which is included herein in its entirety by reference. This involves capturing visual and/or oral data identifying the details of the interaction between physician and patient, e.g., which body regions are being analyzed by the physician or which body regions or other information is provided visually or orally by a patient.

FIG. 2 shows another UI page, which depicts a human skeletal 200. The physician can visually denote a fracture or other bone defect on the depiction of a human skeleton, again by associating an animation from a library 210, with the region of interest on the skeleton 200. This data identifying the fracture of other defect may be provided through DICOM (digital imaging and communications in medicine) imported into the system from X-rays obtained for the patient. The associating of the animation with the region on the skeleton may be performed, for example, by a physician based on the x-ray images received, or may be performed directly by the imaging group by accessing a portal with a UI similar to that described herein.

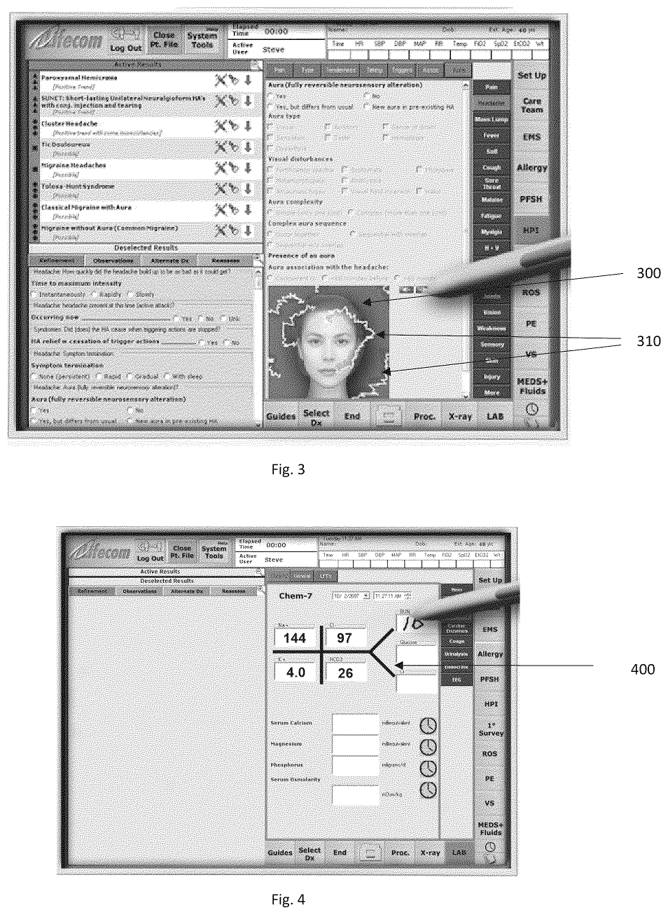

This graphic representation of body parts and systems is also useful in helping to depict a patient's attempt at verbalizing an ailment. In FIG. 3, a migraine is shown graphically, on a depiction of a human head 300, showing the types and location of the pain by means of animations 310, based on an interactive discussion with the patient.

Another use of a graphic representation of information is shown in FIG. 4, which shows the Chem-7 blood profile as depicted using a Y-image 400, which is familiar to clinicians based on how they were taught in medical school, rather than presenting the information in tabular form.

All of these graphic representations provide a quick visual overview of the main issues associated with a patient. In one embodiment, issues that need further attention, e.g., auscultations indicative of a heart murmur, are flagged as risk factors. In one embodiment, when the physician opens a patient's medical record, the physician is immediately presented with a set of postage stamp images associated with flagged events that need to be looked at. The postage stamp images, in one embodiment, are active icons that open up a full depiction of the image when clicked, allowing the risk factor to depicted on the full image. The risk factor, e.g., identifying a heart murmur, may be highlighted on the image of the torso to indicate the location where the heart murmur was identified.

In the above discussion the interaction with the UI was predominantly performed by a clinician such as the physician during a patient encounter or, as discussed above, by a radiologist or x-ray assistant as part of imaging, to depict a fracture or bone defect on the skeleton UI page.

Similarly, other parties, such as the patient could be presented with a portal for purposes of interacting or submitting health-related data. Thus, the present system also serves to improve patient engagement and the patient experience. The patient portal, in one embodiment, is configured to allow the patient to perform patient scheduling, patient self-check-in, self-assessment, as well as provide feedback and respond to surveys in order to improve healthcare. In one embodiment the engagement of the patient may be gamified by allowing the patient to acquire tokens or other incentives for performing tasks and providing feedback. The self-assessment would, for example, allow a patient prior to an appointment, to answer questions or type concerns into a patient portal. The written data can be parsed and mapped onto the depiction of the corresponding body part. Alternatively, the patient could directly associate animations with specific body parts, as was discussed above for FIG. 2. Thus, the patient could, prior to an appointment indicate the location and type of migraine they are experiencing by adding animations to the depiction of the human head.

As is discussed in detail in commonly owned U.S. utility application Ser. No. 17/499,412, in a preferred embodiment, an AI diagnostics engine provides one or more diagnostic results and ranks these together with the source and reasoning for the diagnosis. By basing the diagnosis on CPT codes and ICD-9 and IC-10 codes and generating a physician workflow to support the diagnosis, the system inherently ensures the capture of the workflow steps and the documentation required to support a reimbursement code, thereby avoiding reimbursement denials.

It will be appreciated that while the present invention has been described above with respect to specific embodiments, these have been presented by way of example only and are not intended to limit the scope of the invention as defined by the summary and claims. It will be appreciated that other or additional body parts and systems can be depicted as part of the interactive UI and that the method of interacting with the UI can vary, involving not only the physician but other people, such as the patient, having access to a patient portal with similar graphic depictions.

What is claimed is:

1. An interactive EHR user interface (UI) comprising:
graphic depictions of at least one of: parts of the human body, and systems of the human body, and further comprising:
means for interacting with the depictions of the parts or systems by associating graphical information (also referred to herein as animations) with said depictions of the parts or systems.

2. An interactive EHR UI of claim 1, wherein the graphic depictions of the parts and systems include one or more of a depiction of a human torso, a depiction of a human skeleton, a depiction of a human head, a depiction of a human nervous system, a depiction of a human digestive system, and a depiction of a human circulatory system.

3. An interactive EHR UI of claim 2, wherein one or more of the graphic depictions of the parts and systems include visually defined regions that a physician is required to assess.

4. An interactive EHR UI of claim 3, wherein the animations can be associated with specific locations on the graphic depictions of the parts or systems.

5. An interactive EHR UI of claim 4, wherein the animations are presented as a global library or a separate library for each graphic depiction of a part or system.

6. An interactive EHR UI of claim 5, wherein the process for graphically interacting with the graphic depictions in order to associate the animation with said depictions of the parts or systems, includes selecting an animation (also referred to herein as an icon) from the library of animations and associating it with a specific location or region on or near the depiction of the body part or system.

7. An interactive EHR UI of claim 6, wherein the UI makes use of active controls or drag-and-drop functionality, and wherein associating animations with locations or regions includes using active controls to identify regions and selecting corresponding animations, or by dragging and dropping the animation on the desired location.

8. An interactive EHR UI of claim 7, wherein one or more of image data captured by a video camera, auditory data captured by a microphone, and digital medical data captured by a medical sensor, is processed to provide additional information and to automatically associate animations with identified locations or regions.

9. An interactive EHR UI of claim 1, wherein risk factors identified by a physician or an AI diagnostics engine may be highlighted on the graphic depictions.

10. An interactive EHR UI of claim 9, wherein all body parts and systems identified as having a risk factor, are visually presented when a patient record is opened.

11. An interactive EHR UI of claim 10, wherein the visual representation comprises postage stamp images created as clickable icons that open to a full image with highlighting of the risk factor.

12. A method of reducing reimbursement rejections in medical billing, comprising defining the workflow and potential diagnosis for assisting the physician using an artificial intelligence (AI) diagnostics engine that diagnoses a patient using machine learning in analytics based on medical billing codes.

13. The method of claim 12, wherein the AI engine further includes third-party symptomatic-diagnostic information to diagnose a patient.

14. The method of claim 13, wherein creating and training of predictive models is based on said medical billing codes supplemented with data captured during physician-patient encounters.
